(19) United States
(12) Patent Application Publication
(10) Pub. No.: US 2004/0059197 A1
(43) Pub. Date: Mar. 25, 2004

(54) Connection assembly having communication function and medical apparatus using the connection assembly

(75) Inventors: Seiichiro Yamashita, Kyoto-shi (JP); Kazunari Matoba, Kyoto-shi (JP)

Correspondence Address: KODA & ANDROLIA, 2029 CENTURY PARK EAST, SUITE 1430, LOS ANGELES, CA, 90067-3024, US

(73) Assignee: J. Morita Manufacturing Corporation

(21) Appl. No.: 10/620,107

(22) Filed: Jul. 15, 2003

(30) Foreign Application Priority Data
Jul. 15, 2002 (JP) 2002-206097

(51) Int. Cl. A61B 5/00

(52) U.S. Cl. 600/300

(57) ABSTRACT

A connection assembly detachably connected to a main body of a medical apparatus for use in diagnosis and treatment, wherein the connection assembly has a communication means for sending and receiving the information on said connection assembly to and from said medical apparatus.

### FIELD OF THE INVENTION

The present invention relates to a connection assembly capable of detachably connecting to a main body of a medical apparatus for diagnosis and treatment in a medical or dental field and also relates to a medical apparatus using the connection assembly.

### PRIOR ART

In a medical and dental treatment, many kinds of apparatus are used for several kinds of diagnosis and treatment by detachably connecting to the main body of the medical apparatus.

For example in a dental treatment, there are instruments for diagnosis and treatment to which electricity, water, air and so on is supplied via a flexible tube from the main body, more specifically a cutting device such as a motor handpiece, a turbine handpiece, and so on, a device using ultrasonic or light such as a scaler and a photo polymerization device, and a diagnosis device such as a measuring device of root canal length and an intraoral camera.

Also there are a charging type root canal length measuring device, a chargeable photo polymerization tool, and a intraoral camera as medical apparatus with a self driving source to be controllable and chargeable.

Further, there are devices used for diagnosis or treatment directly or indirectly by detachably connecting to the main body of the medical apparatus, such as a rechargeable battery detachably used for those chargeable devices, a tray table, an instrument holder, an instrument hanger and so on detachable to the main body. Those devices are generically referred as a connection assembly.

Examining a dental instrument as an example of connection assembly, the technical development and evolution of diagnosis and treatment make the diagnosis and treatment method diversify to increase the kinds of instruments. Accordingly, the applicant of the present invention has proposed a multi-junction connection wherein plural instruments are detachably and exchangeable connected to one connection of a main body of the apparatus in JP-A-2000-254153 and JP-A-2000-288001 so that multiple connection parts aren't required to be provided for the apparatus.

Further according to the multi-junction connection, the applicant of the present invention has proposed an identification type instrument for actively outputting a self identification signal for connecting plural instruments to the connection part of the main body of the apparatus in JP-A-2002-35009. FIG. 14 shows one typical embodiment of the identification type instrument.

The instrument 101 is a motor handpiece which is used by detachably and exchangeably connecting to the tip end of the multi-junction tube (not shown) which is a connection part of the main body and has a multi-junction connection part 121 for the connection. The body of the handpiece 101 is comprised of a head 101a for detachably attaching a cutting device T and a middle portion 101b housing a small motor for generating a driving force for rotating the cutting device and also serving as a grip to be grasped by an operator. Identification signal output means 106 for outputting self-identification signal is incorporated in a stator 101c of the small motor and its details are shown in FIG. 14b.

The stator 101c is constructed such that a coil 101ca is provided around an inner cylinder 101cb and is integrally formed by coating and molding with resin 101cc together with a board 101cd mounted with control parts. Connection terminal 101ce is extended from the board 101cd. In this figure, one covered part is broken to show the board 101cd and the coil 101ca, extremely small identification signal output means 106 is arranged together with other control parts on the board 101cd and is integrally covered with resin. Part of the connecting terminals 101ce is used as a connection terminal 106e for the identification signal output means 106.

Upon connecting the instrument 101 to the main body of the apparatus, identification signals are actively output for individually identifying the instrument 101 to the main body of the apparatus from the identification signal output means 106 via the connection terminal 106e. Then, the main body of the apparatus specifies which instrument is connected to supply driving power, water, air and so on corresponding to the connected instrument, thereby setting a display or a control program of the apparatus suitable for the connected instrument. Therefore, different kinds of diagnosis can be executed comfortably by sequentially attaching, detaching and connecting plural kinds of instruments to the same connection part.

According to the above-mentioned multi-junction identification type instrument, although the instrument can be specified, all the control information required for the instrument to be connected has to be prepared on the apparatus side. When the instrument is modified or only an instrument is newly introduced, it has been difficult to prepare the corresponding control information for the apparatus each time specifically for the already installed medical apparatus after sales.

Further, the identification signal output means is electrically connected detachably and exchangeably, therefore it is considered to be housed only in the instrument which directly executes diagnosis and treatment. And it isn't housed in the chargeable battery which is used indirectly and the tray table, the instrument holder, the instrument hanger which aren't electrically connected. Therefore, it hasn't been thought that the identification signal output means is actively used for those connection assembly.

### SUMMARY OF THE INVENTION

The present invention is proposed to solve the above-mentioned problems. The object of the present invention is to provide a connection assembly which doesn't require preparing the corresponding control information on the apparatus side when the connection assembly is modified or changed or a new connection assembly is connected wherein all the connectable parts to the apparatus detachably and exchangeably is considered to be a connection assembly to be positively utilized on the apparatus side.

(1) According to a connection assembly detachably connected to a main body of a medical apparatus for use in diagnosis and treatment of the present invention, the connection assembly has a communication means for sending and receiving the information on the connection assembly to and from the medical apparatus.

This connection assembly is provided with a communication function as the name shows and enables communication to and from the main body of the medical apparatus. In particular it is characterized in that the connection assembly is applied for all the parts which are detachably connected to the main body to be used for diagnosis and treatment and the connection assembly is provided with communication function. The connection assembly includes a cutting treatment device directly used for diagnosis and treatment such as a motor handpiece and a turbine handpiece, a device using ultrasonic or light such as a scaler and a photo polymerization device, an instrument for diagnosis such as a measuring device of root canal length and an intraoral camera, further a detachable charging battery for chargeable device, a tray table, an instrument holder and an instrument hanger which are indirectly used for diagnosis.

When the connection assembly is detached to be exchanged, any information hasn't been obtained from the connection assembly in the prior art. However, because of this communication function, the main body of the medical apparatus can obtain the information from the connection assembly, can execute treatment corresponding to the connected connection assembly and can automate setting of the main body and changing of the control program caused by the exchange, thereby enabling active use of the connection assembly.

(2) According to the connection assembly as set forth in (1), the communication means is comprised of a serial or parallel output type communication means.

(3) According to the connection assembly as set forth in (1) or (2), the communication means has a storage means for memorizing and storing the information on the connection assembly.

(4) According to the connection assembly as set forth in (1)-(3), a microcomputer element or a communication integration element is used as the communication means.

According to the connection assembly, the communication means is specifically defined as a microcomputer element or a communication integration element so that information can be obtained from the connection assembly by communicating with the element in the main body of the medical apparatus, thereby enabling more independent control in the connection assembly. On the other hand, some control may be transferred to the connection assembly and the main body can construct a dispersed processing system.

(5) According to the connection assembly as set forth in (1)-(4), the information is the identification information for identifying the connection assembly and/or the functional information on the function achieved by the connection assembly.

In this connection assembly, the information detail obtained by the communication means is defined and the functional information to be realized by the connection assembly, such as a control program and a display mode can be obtained by the connection assembly itself in addition to the self-identification information as mentioned in the prior art JP-A-2002-35009, for example a back electromotive voltage constant, a torque constant, a current constant, information of display detail relating these constant when the connection assembly is a motor. Therefore, exchange of the connection assembly enables an individual management of the connection assembly and a revision management (compliance with version up of the connection assembly and a newly purchased one) in addition to the specification of the connection assembly, so that it isn't necessary to store the functional information on the new connection assembly in the main body of the medical apparatus.

(6) According to the connection assembly as set forth in (1)-(5), the connection assembly is provided with an identification signal output means and the identification signal output means is provided with a nonvolatile storage means and wherein any one of serial data, voltage level signal of which wave height value is varied at a predetermined repetition cycle, frequency identification signal of which frequency is varied is used as identification signal from the identification signal output means, based on the data stored in the nonvolatile storage means.

The connection assembly defines a generation method of the signals of the information to be communicated by the communication means and the embodiment of the transmission signals. The signals are defined as digitalized signals or digitalizable signals. Accordingly, the connection assembly which can realize the same effects as the connection assembly as mentioned in (1)-(5) can be realized.

(7) According to the connection assembly as set forth in (1)-(6), a connection part for detachably connecting the connection assembly to the main body constitutes a multi junction connection.

The multi-junction connection refers to a connection in which different kinds of connection assembly is designed to be connected to a single connection or any one of the plural connections provided for the main body of the medical apparatus as explained referring to the prior art. The position of the multi-junction connection isn't specified and it may be provided at the tip end of the tube introduced from the main body, if it is directly provided for the main body.

When the connection assembly and the main body of the medical apparatus are connected with such a multi-junction connection, it is important to specify the attached connection assembly and to control so as to achieve the function corresponding to the connection assembly so that the communication means can specify the connection assembly and obtain the functional information.

(8) According to the connection assembly as set forth in (1), the communication means is a passive element electrically connected to the main body of the medical apparatus.

This connection assembly doesn't actively output the information to be communicated with the main body of the medical apparatus. It incorporates an resistive element such as resistance therein and the resistance value of the resistive element is read as the information of the connection assembly in the main body so that the communication means can be simply constructed.

(9) According to the connection assembly as set forth in (1)-(8), the connection assembly includes a charging battery.

In this embodiment, the connection assembly provided with a charging battery is used as an example of the connection assembly as mentioned in (1) which hasn't had a communication means in the prior art. The communication means is provided for the charging battery so that the connection assembly has communication function. The charger being a main body of the medical apparatus can obtain the information such as the battery volume and battery voltage of the charger being a connection assembly, thereby enabling appropriate battery charge for the connected charging battery and requiring no preparation of the charging battery per different charging batteries.

(10) According to a medical apparatus for use in diagnosis and treatment in which a connection assembly is detachably connected to a main body of the medical apparatus, the connection assembly is comprised of a communication means for sending and receiving information on the connection assembly to and from the main body of the medical apparatus, and function to be realized by the connection assembly is realized cooperating with the connection assembly by the information obtained from the communication means upon connecting the connection assembly to the main body of the medical apparatus.

This medical apparatus is designed such that the connection assembly with communication function is detachably connected, the information is obtained from the connection assembly, and the function to be achieved by the connection assembly is realized. Therefore, the main body of the medical apparatus can comply with each connection assembly without storing the functional information per the connection assembly in advance in the main body.

(11) According to the medical apparatus for use in diagnosis and treatment as set forth in (10), a part of the function is realized by setting a driving circuit or a control circuit corresponding to the connected connection assembly.

This medical apparatus sets the drive circuit and the control circuit according to the information sent from the connection assembly so that an operator can execute operation corresponding to the connected connection assembly without selecting each drive circuit.

(12) According to the medical apparatus for use in diagnosis and treatment as set forth in (10) or (11), a part of the function is achieved by setting display mode of display means and/or input mode of input means such as a touch panel corresponding to the connected connection assembly.

In this medical apparatus, the display mode of the display means and the input mode of the input means can be complied with the information sent by the connection assembly, thereby enabling an operator to display and operate according to the connected connection assembly without selecting each display mode.

(13) According to the medical apparatus for use in diagnosis and treatment as set forth in (10)-(12), the management of usage history and the distinction of using operator of the specified connection assembly can be executed, when the connection assembly is specified based on the information obtained from the connection assembly.

The medical apparatus can identify the connection assembly by the identification information communicated from the connection assembly, thereby executing an individual management such as usage history of the connection assembly and more minute management.

(14) According to the medical apparatus for use in diagnosis and treatment as set forth in (10)-(13), the main body of the medical apparatus has a microcomputer element or a communication integration element as a communication means for connecting the connection assembly.

In this medical apparatus, the communication means of the information communicated from the connection assembly is defined. Because the communication means of the connection assembly actively outputs necessary information, a microcomputer element or a communication integrated element may be provided as an identification means of the main body of the medical apparatus so that the apparatus is simply constructed. Further, if a microcomputer element is already provided as a control device of the main body, it may be used as an identification means by revising its software.

(15) According to the medical apparatus for use in diagnosis and treatment as set forth in (10)-(14), wiring to a connection part for detachably connecting the connection assembly in the main body constitutes a multi-branch structure.

The multi-branch structure refers to a construction like a tree in which assuming the place from which parts are connected the root of a tree, each place to be connected is like a branch of the tree as shown in FIG. 9c.

This medical apparatus utilizes the advantage such that the attached connection assembly actively outputs information so that the passing route doesn't affect identification of the connection assembly. According to this apparatus, the connection to the connection part of the connection assembly isn't separated like in the prior art and it is cleared that the multi-branch structure is possible, thereby simplifying the construction of the medical apparatus.

(16) According to the medical apparatus for use in diagnosis and treatment as set forth in (10)-(15), the connection assembly is the connection assembly with communication function as set forth in (1)-(9).

According to this embodiment, it is cleared that the medical apparatus as mentioned in (10)-(15) is complied with the connection assembly with communication function in (1)-(9) and each effect mentioned above is synergistically realized.

### BRIEF DESCRIPTION OF THE DRAWINGS

FIG. 1a is an external perspective view exemplifying a medical apparatus using a connection assembly having communication function of the present invention, FIG. 1b is a detailed view with parts broken away of a connection part of the main body of the medical apparatus to which the connection assembly is detachably connected, FIG. 1c is a circuit diagram of the connection part of the main body shown in FIG. 1b, and FIG. 1d is a circuit diagram of the connection assembly shown in FIG. 1b.

FIG. 2 shows a basic concept of an essential part exemplifying a connection assembly with communication function and a medical apparatus using the connection assembly according to the present invention.

FIG. 3 shows a basic concept of an essential part exemplifying a connection assembly with communication function and a medical apparatus using the connection assembly according to the present invention.

FIG. 4 shows a basic concept of an essential part exemplifying a connection assembly with communication function and a medical apparatus using the connection assembly according to the present invention.

FIG. 5 shows a basic concept of an essential part exemplifying a connection assembly with communication function and a medical apparatus using the connection assembly according to the present invention.

FIG. 6a is an information flow diagram showing the information flowing between the connection assembly and the main body of the medical apparatus shown in FIG. 5 and FIG. 6b shows a sample of a display screen.

FIG. 7 is a flow chart showing the operation of the main body when the connection assembly of the present invention is connected.

FIG. 8 shows a table α1 used for setting torque and rotation number in the present invention.

FIG. 9 explains a communication means used for the connection assembly having communication function according to the present invention. FIG. 9a is a block diagram conceptionally showing the construction, FIG. 9b is a conceptional diagram showing the connection embodiment of the main body and signal, FIG. 9c is a conceptional diagram showing a multi-branch structure of the main body for connecting the communication means, and FIG. 9d is a time chart exemplifying the signal output from the communication means.

FIG. 10a is a partially broken front view showing other embodiment of the connection assembly with communication function according to the present invention and FIG. 10b is an external perspective view of the partially broken stator incorporated in the communication means.

FIG. 11 shows a block diagram conceptionally showing one embodiment of the medical apparatus using the connection assembly with communication function.

FIG. 12 shows a block diagram conceptionally showing other embodiment of the medical apparatus using the connection assembly with communication function.

FIG. 13 shows a conceptional diagram showing other embodiment of the connection assembly with communication function according to the present invention.

FIG. 14a is a partially broken front view showing an identification type instrument in the prior art, FIG. 14b is a partially broken perspective view of the stator shown in FIG. 14a.

### DETAILED DESCRIPTION OF THE INVENTION

Here the embodiments of the present invention are explained referring to the attached drawings. Dental connection assembly and medical apparatus are explained hereinafter as an example, however, the filed of the invention isn't limited to these areas as mentioned above.

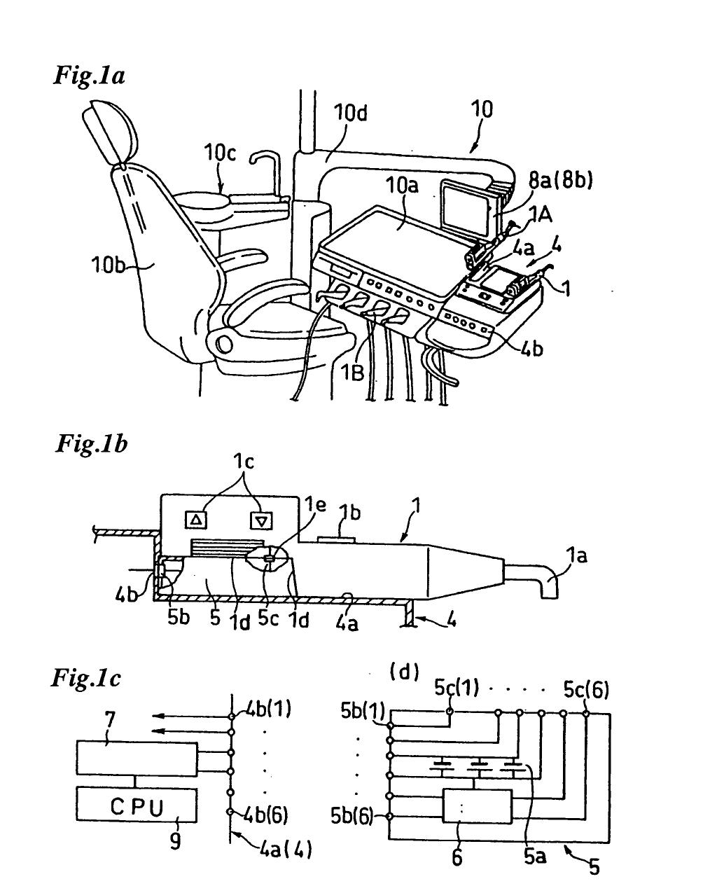

FIG. 1a is an external perspective view exemplifying a medical apparatus using a connection assembly having communication function of the present invention, FIG. 1b is a detailed view with parts broken away of a connection part of the main body of the medical apparatus to which the connection assembly is detachably connected, FIG. 1c is a circuit diagram of the connection part of the main body shown in FIG. 1b, and FIG. 1d is a circuit diagram of the connection assembly shown in FIG. 1b.

The medical apparatus 10 executes dental diagnosis and treatment in such a manner that instruments (connection assembly) 1 and 1A with communication function are connected to a connection part 4. The apparatus 10 has a treatment bed 10b for executing treatment of a patient thereon, a basin table 10c provided beside the treatment bed 10b for supplying gargle water to a patient and for receiving discharged water after gargling, a side table support arm 10d, and a side table 10a supported by the support arm 10d.

A display means 8a also serving as an input means 8b is provided for the side table 10a in order to be easily seen by an operator working at the side table 10a. Connection part 4 is provided for the side table 10a and plural instruments 1B connected with flexible tubes are held where they can be easily taken in and out.

The side table 10a communicates with the instrument (connection assembly) 1 with communication function via the connection part 4 and incorporates a communication control means (CPU) 9 for entirely controlling the apparatus 10 so as to be functioned as an apparatus body 10a.

The instrument (connection assembly) 1 is a cordless type photo polymerization device provided with a detachable and exchangeable charging battery 5 and has a probe 1a for emitting light, a power button 1b for turning on and off the light, a control button 1c for controlling the light volume, and a battery installation part 1d for detachably installing the charging battery 5 and having a connection terminal 1e.

The charging battery 5 has a communication means 6 for sending and receiving information relating to the charging battery with the main body 10a, a charging part 5a chargeable by means of outer power source, a connection terminal 5b (1-6) for electrically connecting with the connection part 4 and a connection terminal 5c (1-6) corresponding to the connection terminal 1e of the instrument 1.

Each connection terminals 5b and 5c is comprised of the terminals 5b (1, 2) and 5c (1, 2) for communicating and controlling between the main body of the medical apparatus 10a and the main body of the instrument 1, terminals 5b (3, 4) and 5c (3, 4) for charging and discharging electricity, and terminals 5c (5, 6) for communicating the charging battery and the main body of the instrument 1. The terminals 5b and 5c(4) are used as a common contact terminal for the charging battery 5a and the communication means 6.

The instrument 1A is a measuring device of root canal length which is a cordless type like the instrument 1, and has a charging battery, but the battery volume and voltage are different from those of the instrument 1.

The connection part 4 has a charge control circuit 7 to be connected to the charging battery 5, is connected with the communication control means (CPU) 9 which is also communicated with the communication means 6 of the charging battery 5, is provided with a receiving part 4a for receiving and holding each instrument 1 and 1A. The receiving part 4a has a connection terminal 4b (1-6) corresponding to the connection terminal 5c (1-6) of the charging battery 5.

The receiving part 4a can receive and hold any one of cordless type instruments (such as an intraoral camera) other than the instruments 1, 1A. It is important to determine which instrument is attached and to charge suitable electricity. In such a case the instrument (connection assembly) and the charging battery (connection assembly) with communication function according to the present invention realizes their effects.

Namely, the instrument 1 has the charging battery 5 incorporating the communication means 6. Only by being received and held in the receiving part 4a of the connection part 4, the main body 10a of the medical apparatus can obtain information on the charging battery 5 such as battery volume and battery voltage by communicating with the communication means 6, thereby enabling to charge electricity suitably by instructing the charge control circuit.

The main body of the medical apparatus can obtain information from the instrument constituting a connection assembly, namely a charging battery, by the communication function. Therefore, treatment corresponding to the connected connection assembly is possible, and setting of the main body and change of the control program caused by attaching even after detaching or exchanging instruments can be automatically done, so that the connection assembly can be actively utilized.

In this case, the charging battery 5 itself has the communication means 6 so that even when it is separated from the main body, it can bring out its communication function and be appropriately charged with electricity while being held on the receiving part 4a of the connection part 4. Therefore, while using the instrument 1 with the charged battery 5 attached, an auxiliary charging battery 5 may be attached on any one of the receiving part 4a, which isn't used, of the connection part 4 to be charged and the battery 5 may be exchanged if necessary.

Further, the connection part 4 can be attached with other cordless type instruments and the charging battery for these instruments, thereby facilitating automatic battery charge corresponding to the battery volume and battery voltage.

The main body of the instrument 1 is also the main body for the charging battery 5 and has a communication control means (CPU) 9 similar to that of the main body 10a. When the charging battery is detached and exchanged, the instrument 1 receives the information on the charging battery and determines whether an appropriate charging battery is attached or not. If an improper charging battery is attached, it announces an alarm requesting exchange to an operator.

Further, such a connection assembly (instrument and charging battery) with communication function is available so that the medical apparatus can achieve the effect of the connection assembly as a medical apparatus.

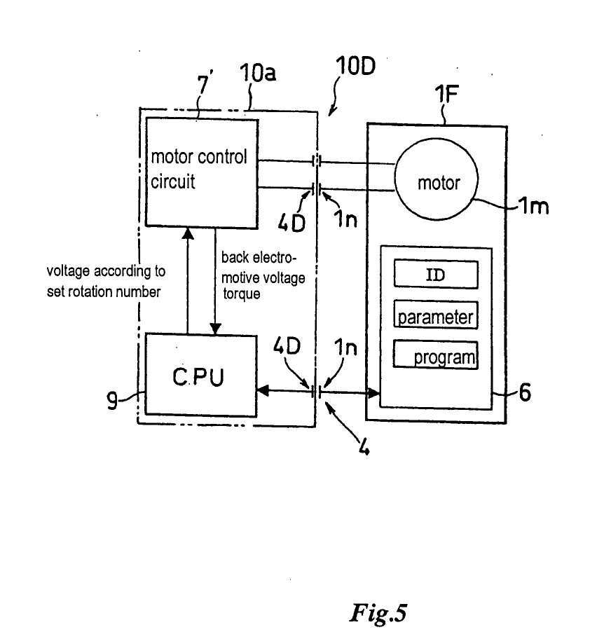

The instrument 1B connected with a flexible tube is also a connection assembly with communication function and its details are explained referring to FIG. 5.

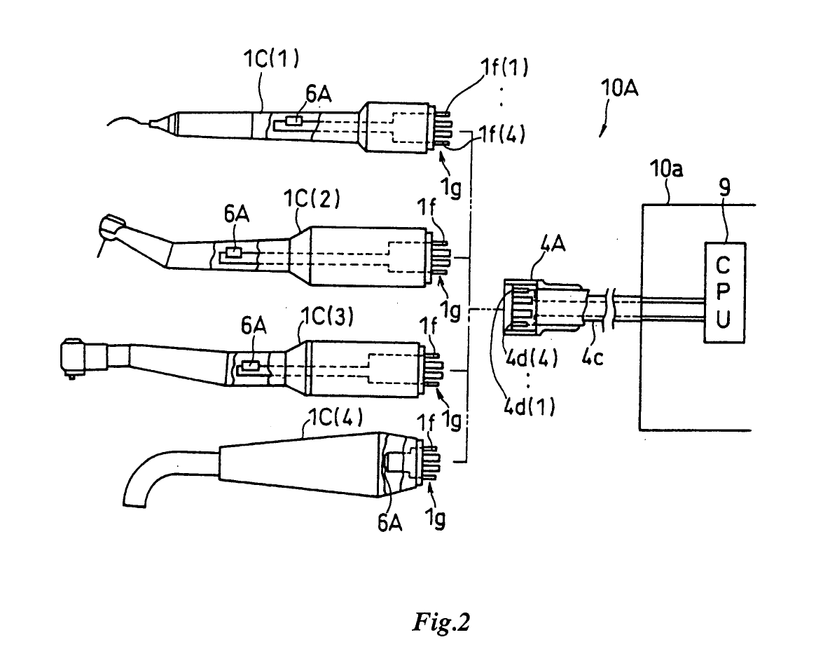

FIG. 2 shows a basic concept of an essential part exemplifying a connection assembly with communication function and a medical apparatus using the connection assembly according to the present invention. The members already explained have the same reference numbers and its explanations are omitted hereinafter.

According to the medical apparatus 10A, the instrument 1C (1-4) constituting a connection assembly with communication function is detachably and exchangeably connected to the connection part 4A provided at the tip end of a flexible tube 4c introduced from the main body 10a of the medical apparatus. Each instrument 1C works as a scaler 1C(1), a motor handpiece 1C(2), a motor handpiece 1C(3) and a photo polymerization device 1C(4).

Each one of the instruments 1C (1-4) has a simple resistive element as a passive element as a communication means 6A for sending and receiving the information on each instrument with the main body 10a and the communication means 6A. The resistance value of the resistive element is different from each instrument.

Connection part 1g for connecting the main body 1a and the connection part 4A is provided and is a multi-junction connection capable of connecting any instrument 1C (1-4) to one connection part 4A. The connection part 4A of the main body 10a is correspondingly constructed as a multi-junction type.

Common connection terminal if (1-4) is provided for the connection part 1g and the connection terminals If(1) and 1f(4) are for communicating with the communication means 6A. Correspondingly, the connection part 4A of the main body 10a is provided with connection terminal 4d (1-4).

When any one of the instrument 1C (1-4) is connected to the connection part 4A, the apparatus body 10a reads the resistance value of the communication means 6A from the communication control means 9 via the connection terminal 4d (1, 4). Thereby, the main body 10a determines which instrument 1C (1-4) is connected or determines the control detail corresponding to the connected instrument and a drive circuit, a control circuit or a display mode can be controlled.

Therefore, a passive element which is a simple resistance can be used as a communication means of the present invention and the same effect can be achieved in the connection assembly and the medical apparatus.

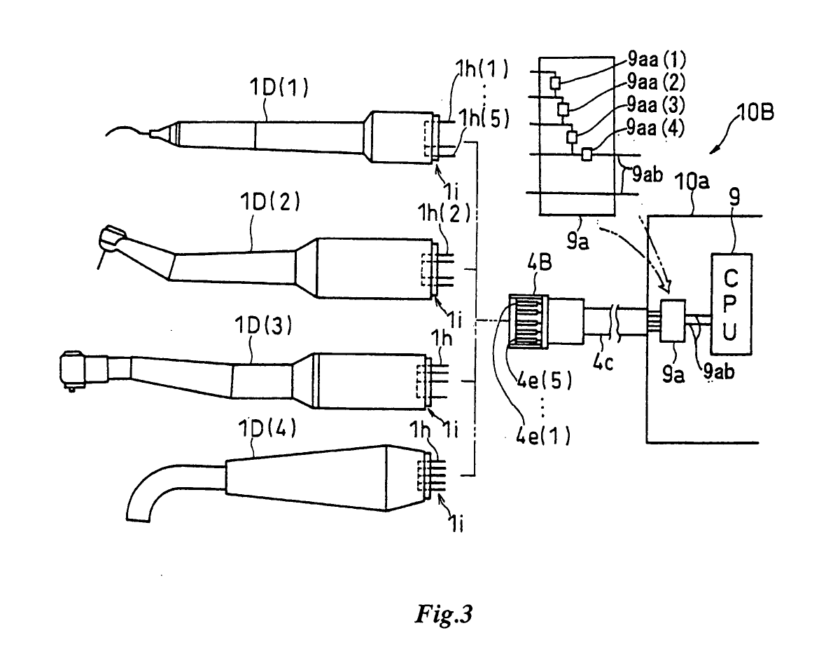

FIG. 3 shows a basic concept of an essential part exemplifying a connection assembly with communication function and a medical apparatus using the connection assembly according to the present invention.

In the medical apparatus 10B the instrument 1D (1-4) constituting a connection assembly with communication function is detachably and exchangeably connected to a connection part 4B provided at the tip end of a flexible tube 4c introduced from the main body 10a of the medical apparatus like the medical apparatus 10A and works as a scaler 1D(1), a motor handpiece 1D(2), a motor handpiece 1D(3) and a photo polymerization device 1D(4).

Communication means 6A isn't incorporated in each one of the instruments 1D(1-4) like the instrument 1C. The instrument 1D (1-4) is different from the instrument 1C in that a communication terminal 1h (1-5) for communicating information is selectively provided corresponding to each instrument at a multi-junction connection 1i thereof in addition to a communication terminal for drive control (not shown) and the selectively provided communication terminal 1h is connected each other.

Correspondingly in the main body 10a, the multi-junction connection part 4B is provided with a corresponding communication terminal 4e (1-5) and further a logical circuit incorporating a logical element 9aa (1-4) which is connected to the communication terminal 4e (1-5) respectively is provided. Output of the logical circuit 9a is connected to the communication control means 9 via a signal line 9ab.

In such a construction, when any one of instrument 1D (1-4) is connected to the connection part 4B, the incorporated logical element in the logical circuit 9a is connected in a different manner corresponding to the connected instrument 1D. For example, if the instrument 1D (1) is connected, only the logical element 9aa (1) and 9aa (4) are connected, the communication control means 9 determines which one is connected among the instruments 1D (1-4) or determines the control corresponding to the connected instrument, so that a driving circuit, a control circuit, or a display mode can be controlled according to the instrument.

Thus, such a resistive element, which is only a simple element arrangement, can be used as a communication means of the present invention, thereby achieving the same effect in the connection assembly or in the medical apparatus.

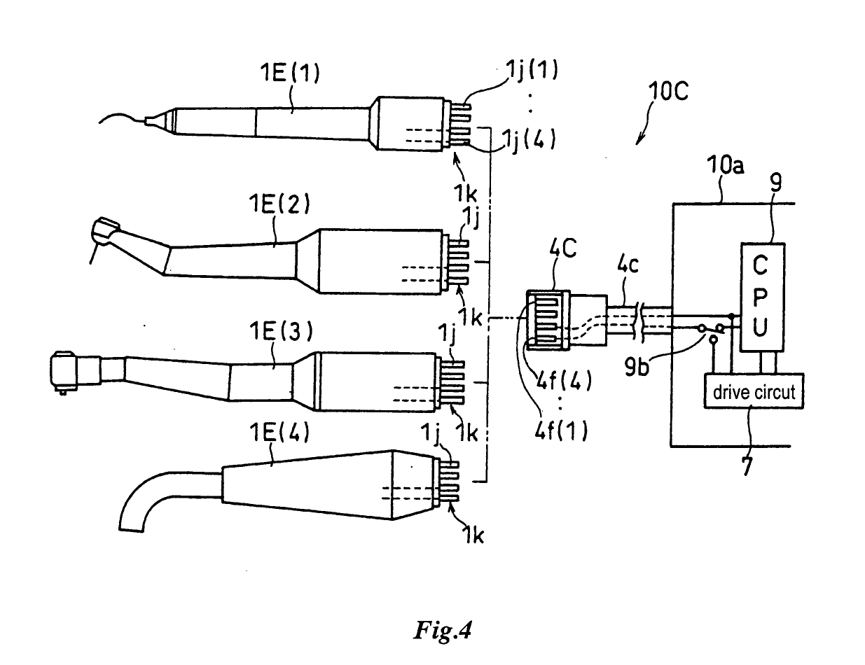

FIG. 4 shows a basic concept of essential part exemplifying a connection assembly with communication function and a medical apparatus using the connection assembly according to the present invention.

In the medical apparatus 10C the instrument 1E (1-4) constituting a connection assembly with communication function is detachably and exchangeably connected to a connection part 4C provided at the tip end of a flexible tube 4c introduced from the main body of the medical apparatus 10a like the medical apparatus 10A and works as a scaler 1E(1), a motor handpiece 1E(2), a motor handpiece 1E(3) and a photo polymerization device 1E(4).

Communication means 6A isn't incorporated in each one of the instruments 1E(1-4) like the instrument 1C. The instrument 1E (1-4) is different from the instrument as mentioned above in that an information terminal 1h isn't provided like the instrument 1D and the instrument 1E is constructed the same as the prior instrument without communication function. However, each instrument 1E (1-4) is limited to one using electricity at least one part thereof.

Connection terminal 1j (1-4) for drive control is provided for a multi-junction connection 1k of the instrument 1E (1-4) and the connection terminal 1j (3, 4) are used as a terminal for electric connection.

Corresponding connection terminal 4f (1-4) is provided for the multi-junction connection 4C of the main body of the medical apparatus 10a.

In the medical apparatus 10C, because such a normal instrument 1E (1-4) is used as an instrument (connection assembly) having communication function, it is different from the prior medical apparatus in that a communication drive switch means 9b is provided for switching connection of the electric connection terminals 4f (3,) into either one of a drive circuit 7 or a communication control means 9. The connection terminal 4f (3) is a contact terminal.

According to the medical apparatus 10C, when any one of the instrument 1E (1-4) is connected to the connection part 4C, the communication drive switch means 9b is set to be communication side so that the connection terminal 4f (3, 4) and the communication control means 9 are connected in the main body of the medical apparatus 10a. In the communication control means 9, the resistance or impedance between the connection terminals 4f (3, 4) is measured and which one of the instruments 1E (1-4) is connected is determined or the control detail corresponding to the connected instrument is determined.

Then, the communication drive switch means 9b is switched to the drive circuit 7 and the communication control means 9 can control the drive circuit, the control circuit or the display mode corresponding to the connected instrument.

According to the medical apparatus 10C, the electric load itself of the instrument is used as a communication means being a resistive element by providing the communication drive switch means 9b.

In the instrument using electricity as mentioned above, its electric load becomes a resistive element without providing particular devices, thereby available for the communication means of the present invention and achieving the same effect in the connection assembly and the medical apparatus.

In FIG. 2-FIG. 4 examples of communication means as a resistive element are shown. However, it is most important in the present invention the inventors have found the information by these resistive elements is not only used as an identification and specification means of the instrument which is a detachable connection assembly but also used as a control information for directly controlling the connected instrument.

Accordingly the present invention is characterized in that the communication means as a resistive element doesn't communicate an identification information for identifying the connected instrument but communicates a functional information such as parameter relating to the rotation number and output of the motor of the connected instrument. Of course in this case, the identification information may be communicated in addition to the functional information. It is also important that the communication means is provided also for the connection assembly which is detachably connected to the main body of the medical apparatus.

When the connection assembly which is detachably and exchangeably used has several parameters, is changed, or modified and revised, the functional information on the connection assembly is contained in the communication means incorporated in the connection assembly itself and the functional information is sent to the main body only by connecting the connection assembly to the main body. When the main body receives the information, treatment and diagnosis can be executed using the exchanged connection assembly without preparing the functional information on the connected assembly in the main body in advance. For example in case of a motor handpiece, plural kinds of motor handpiece with different rotation numbers can be appropriately used depending on purpose.

FIG. 5 shows a block diagram exemplifying a connection assembly with communication function and a medical apparatus using the connection assembly according to the present invention.

In the medical apparatus 10D, the instrument 1F which is a motor handpiece is detachably and exchangeably connected to the main body 10a of the medical apparatus as a connection assembly. Its practical construction is the same as that shown in FIG. 1, FIG. 2, FIG. 3 and FIG. 4 so that the difference between those figures is explained hereinafter.

The instrument 1F has a motor lm being a drive source, a communication means 6, and a connection part in for detachably and exchangeably connecting to the main body of the medical apparatus 10a. ID as an identification information, parameter and program as a functional information on control, are stored in the communication control means 6 as the information on the instrument 1F.

The main body 10a has a motor control circuit 7′, a communication control means (CPU) 9, and a connection part 4D for detachably and exchangeably connecting the instrument 1F in which the motor control circuit 7′ and the communication control means 9 are communicated each other, for example back electromotive voltage torque in proportion to the rotation number of the motor is sent to the communication control means 9 from the motor control circuit 7′ and the voltage corresponding to the set rotation number of the motor is sent in reverse.

When a connection part In of the instrument 1F is connected to the connection part 4D of the main body 10a, a motor 1m of the instrument 1F and the motor control circuit 7′ of the main body 10a are connected, and the communication control means 9 and the communication means 6 are connected.

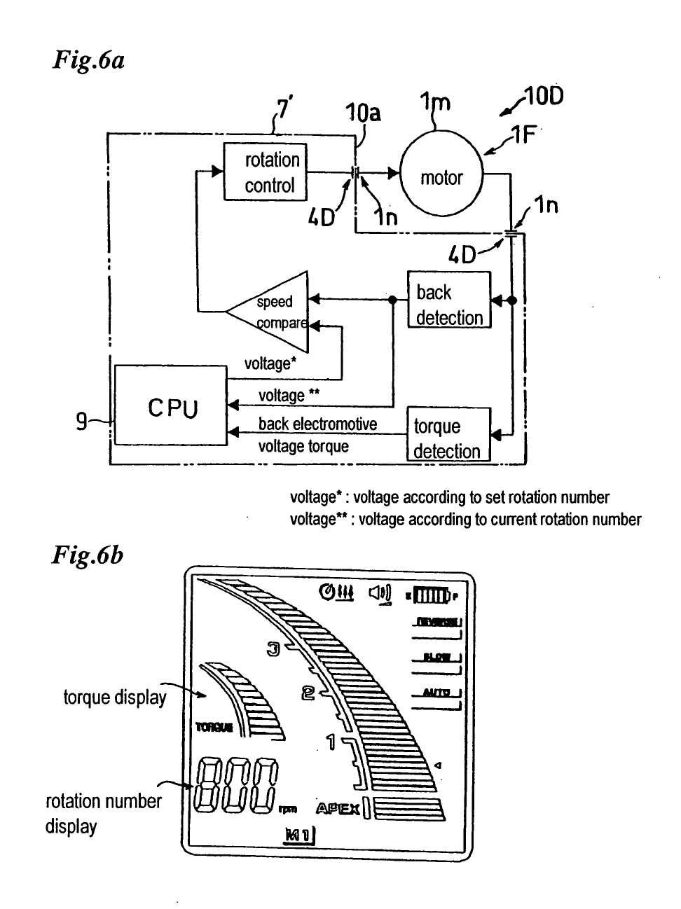

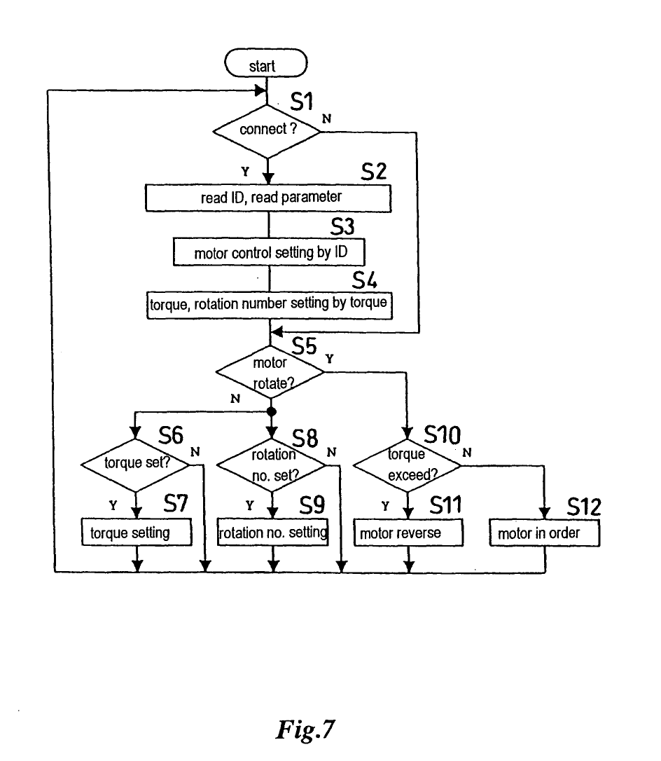

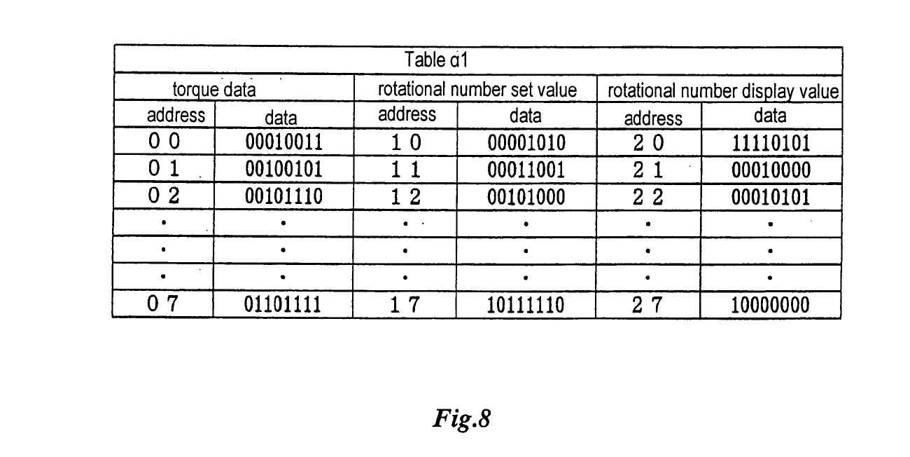

FIG. 6a is an information flow diagram showing the information flowing between the connection assembly and the main body of the medical apparatus shown in FIG. 5 and FIG. 6b shows a sample of a display screen. FIG. 7 is a flow chart showing the operation of the main body when the connection assembly of the present invention is connected and FIG. 8 shows a table α1 used for setting torque and rotation number in the present invention.

In the main body of he medical apparatus 10a, the connected condition of the instrument is always observed. Immediately after confirming the instrument 1F is connected to the main body 10a (S1), the communication control means 9 reads in ID which is an identification information on the motor lm, and parameter being functional information on control, such as a back electromotive voltage constant for controlling the rotation number of the motor in order to change the control of the motor control circuit 7′ and torque constant for controlling the torque in response to the motor load and a predetermined rotation number, torque and these programs, from the communication means 6 (S2). Based on the identification information and/or the functional information, display control and drive control for the instrument 1F are executed (S3) and the set rotation number and the set torque value of the motor lm are determined from the parameter included in the functional information (S4).

FIG. 6b shows such a display mode on a display means 8a also serving as the input means 8b explained referring to FIG. 1 in which the torque display and the rotation number display correspond to the instrument 1F.

Here the motor lm is monitored to be rotated or not (S5), if rotating, the torque is detected and whether the torque exceeds the set toque obtained as mentioned above or not is determined (S10), if exceeding, the motor lm is rotated in reverse (S11) and if not, the motor lm is rotated in order (S12).

When the motor 1m is stopped, the rotation number and the torque value are set according to the parameter value read in the procedure (S4) in the medical apparatus 10D, for example, it is executed according to the table α1. In the table the address means where the data is stored.

Supposing the parameter read in the procedure (S4) is the same as the table α1, wherein the torque, the rotation number set value and the rotation number display value are shown from the left. When the torque set is indicated in the procedure (S6), an optional parameter (data) is selected by means of a selection switch (not shown) from those read in the address 00-07, thereby setting the torque (S7).

“00010011” which is set for the address 00 in case of torque 0 and “00101110” which is set for the address 02 in case of torque 2 are set also in the main body of the medical apparatus. In this case, the address value and the name of the torque setting are accorded for easy understanding, however, it is only one example.

It is also the same as the rotation number setting. When the rotation number set is indicated, an optional data is selected among the data read in the address 10-17 to set the rotation number (S9). Otherwise, the data of the rotation number display value may be read from the address 20-27 and the rotation number display according to the rotation number setting may be shown on the display means 8a.

Such setting is possible for each one of plural instruments 1F so that different control program per the instrument isn't required to be prepared and different parameter can be set for the same instrument. Therefore, it is flexible that the rotation number and the torque may be controlled according to the operator's request/(Also in this case, the control program of the communication control means 9 isn't required to be changed.)

Further explained is the case when a program code is read from the communication means 6. Generally, the instrument can be flexibly controllable only by changing the parameter, however in some cases, it may not be corresponded only by changing the parameter.

In such a case, it is obliged to drastically change the hardware in the prior art, however in the present invention, the program code for controlling the instrument, which isn't executed by changing the parameters, is obtained from the communication means 6 of the instrument itself, thereby enabling to control the instrument by the program code. Therefore, when it isn't complied by the parameter change, the instrument is flexibly controlled without changing the hardware.

Thus set rotation number and torque are sent to the communication means 6 of the instrument 1F from the main body 10a of the medical apparatus to revise, update and store the rotation number and torque which have been already stored in a rewritable storage means (for example EEPROM) incorporated in the communication means 6. Therefore, different set rotation numbers and torques may be prepared for plural instruments 1F, thereby being selectively usable by each operator's request.

The instrument 1F which stores parameters such as revisable set rotation number and torque as functional information in the communication means 6 is preferable as a measuring device of root canal length which requires minute adjustment of the rotation number and the torque and also requires correct reverse rotation at a predetermined torque.

Generally different motors are mounted for each instrument, but any motor can be complied by adjusting and changing the circuit constant depending on the functional information without changing the circuit even when the performance and specification are different. Further, when the kinds of the instrument are different, the switch for displaying and setting is usually different, however in the present invention, the setting of the display mode and the switch are possible per each instrument according to the parameter and the program code sent by the communication means 6, thereby flexibly available for operation environment.

The main body in this specification includes a unit and a control box which are detachably installed for the medical apparatus and are used for partial control.

Further the information required for control is transmitted to the main body 10a via the communication means 6 consisting of a microcomputer mounted on the connection assembly 1 or is obtained by reading the memory (communication means 6) incorporated in the connection assembly 1 from the main body 10a.

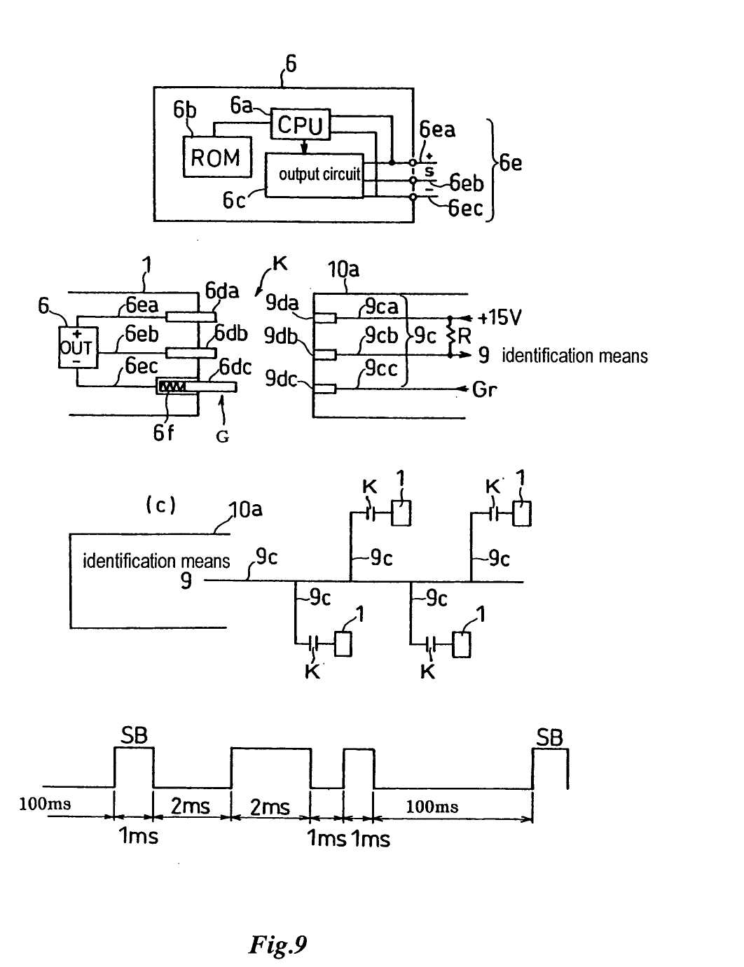

FIG. 9 explains a communication means used for the connection assembly having communication function according to the present invention. FIG. 9a is a block diagram conceptionally showing the construction, FIG. 9b is a conceptional diagram showing the connection embodiment of the main body and signal, FIG. 9c is a conceptional diagram showing a multi-branch structure of the main body for connecting the communication means, and FIG. 9d is a time chart exemplifying the signal output from the communication means. In these figures the communication means, the apparatus body and the connection therebetween which have been explained hereinbefore are generally explained.

The communication means 6 in FIG. 9a is one example of the communication means used in the present invention and is comprised of CPU 6a for entirely controlling the output means 6, a memory means 6b for memorizing the information on the incorporated connection assembly, an output circuit 6c to be activated by the instruction from CPU 6a, and a connection wire constituting an power supply line 6ea, a signal line 6eb, and a common line 6ec. The memory means is constructed with a nonvolatile memory ROM, a memory which is rewritable and capable to memorize and store by itself (for example EPROM), and if necessary.

The communication means 6 is incorporated in the connection assembly 1 with communication function as shown in FIG. 9b and connection terminals 6da, 6db and 6dc are provided for the connection assembly 1 corresponding to each one of the connection wires 6e. The connection assembly 1 is detachably attached to the main body 10a.

Terminal receivers 9da, 9db and 9dc are provided for the main body corresponding to each connection terminal 6da, 6db and 6dc of the connection assembly 1 and are connected to the electric source line 9ca, the signal line 9cb and the common line 9cc respectively in which a resistance R is bridged between the electric source line 9ca and the signal line 9cb.

These electric source line 9ca, signal line 9cb and common line 9 cc are collectively called a connection wire 9c of the main body. The signal line 9cb is connected to an identification means 9 installed in the main body. In this example, the connection part between the connection assembly 1 and the apparatus body 10a is referred to K.

When the connection assembly 1 is attached to the main body 10a, the connection terminals 6da, 6db, 6dc of the connection assembly 1 are connected to the terminal receivers 9da, 9db, 9dc of the main body 10a respectively so that electric power (direct current at 5V in this embodiment) is supplied from the apparatus body. The CPU 6a of the communication means 6 detects the supply, reads out the information on the connection assembly 1 from the memory means 6b, and outputs the information from the signal 6eb via the output circuit 6c. Upon receiving the identification signal by the signal line 9 db, the identification means 9 simply reads in the output information or communicates with the communication means 6 to specify the connection assembly 1 or to execute control corresponding to the connection assembly 1. Other operations are the same as the mentioned hereinbefore.

Here the connection terminal 6dc of the connection assembly 1 is a contact terminal slidable back and forth and is provided with a spring 6f therein so as to be projected forward comparing with other connection terminals 6da and 6 db by the spring.

Therefore, for connecting at the connection part C, the connection terminal 6dc is connected to the terminal receiver 9dc of the other at first, then, while keeping the connection, the other terminals 6da and 6 db are connected to the terminal receivers 9da and 9 db. So that other connection terminals than the contact terminal 6dc is prevented from being connected by mistake, thereby eliminating adverse effect on the connected elements.

The communication means 6 may be comprised of the above-mentioned CPU 6a with simplest construction which receives electricity from the main body and mutually communicates the serial or parallel output of the information on the connection assembly 1 as identification signal, thereby achieving the simplest communication means.

However, the communication means 6 may be constructed with the CPU 6a as a high performance microcomputer element or communication integration element. In such a case the connection assembly can execute more independent control, on the other hand in the main body, a dispersed processing system can be constructed by transferring some control to the connection assembly.

The above information is read out of the memory means 6b and voltage level signals of which wave height value is varied at a predetermined repetition cycle, or frequency identification signals of which frequency is varied are used other than the serial data mentioned hereinbefore.

Because the communication signal 6 actively outputs required information, the passage route of the signal doesn't affect on specifying the connection assembly. So that the connection when plural connection parts C are provided may be a multi-branch structure like FIG. 9c, thereby simplifying the wiring of the main body without providing separate wirings in the prior part.

Further explained hereinafter referring together with FIG. 9d is a communication method wherein connection to the connection part C is a multi-branch structure, ID code output element is used as a communication means 6 and ID code which is a serial data is output as identification formation.

When nothing is connected to the main body 10a, the signal line 6eb is always “H” and the identification means 9 detects as no-connection and shows for example “no-connection”.

When the connection assembly 1 incorporating a communication means 6 is connected, an electricity is supplied from the main body 10a and the communication means 6 outputs serial data as shown in FIG. 9d. The data is constructed with the combination of “L” more than 100 ms, “H” or “L” at 1 ms and “H” or “L” at 2 ms to send a code (001101B) with 6 bit per 1 ms. The “H” signal with 1 ms after “L” above 100 ms is defined as a start bit SB.

The communication means 6 continuously outputs such serial data and upon receiving the data the identification means 9 of the main body 10a samples the received data per 1 ms to detect data, and reads in the ID code of the connected connection assembly, thereby specifying the connection assembly 1.

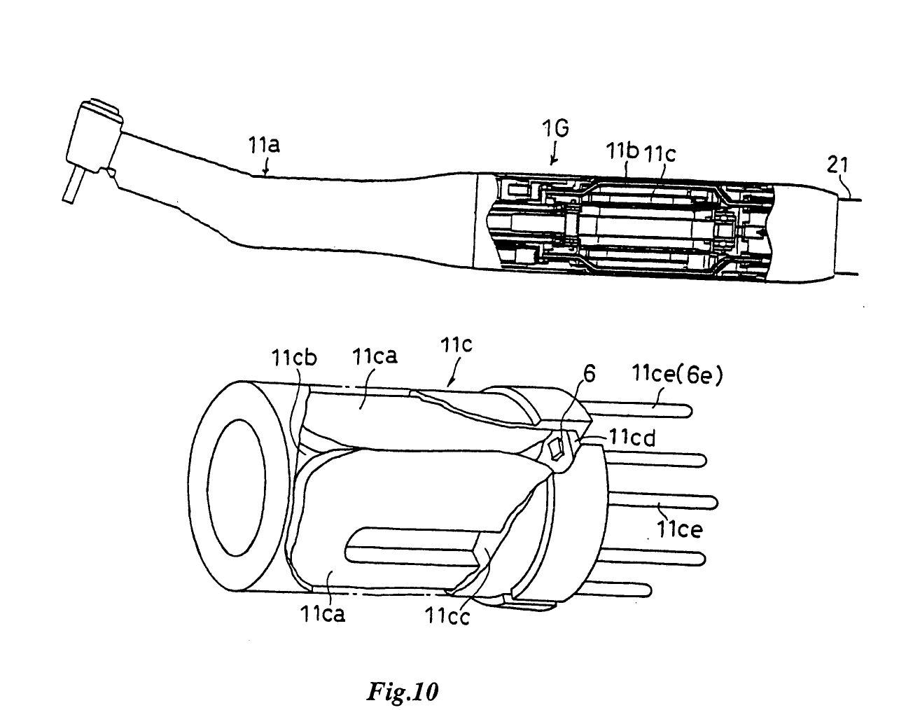

FIG. 10a is a partially broken front view showing other embodiment of the connection assembly with communication function according to the present invention and FIG. 10b is an external perspective view of the partially broken stator incorporated in the communication means.

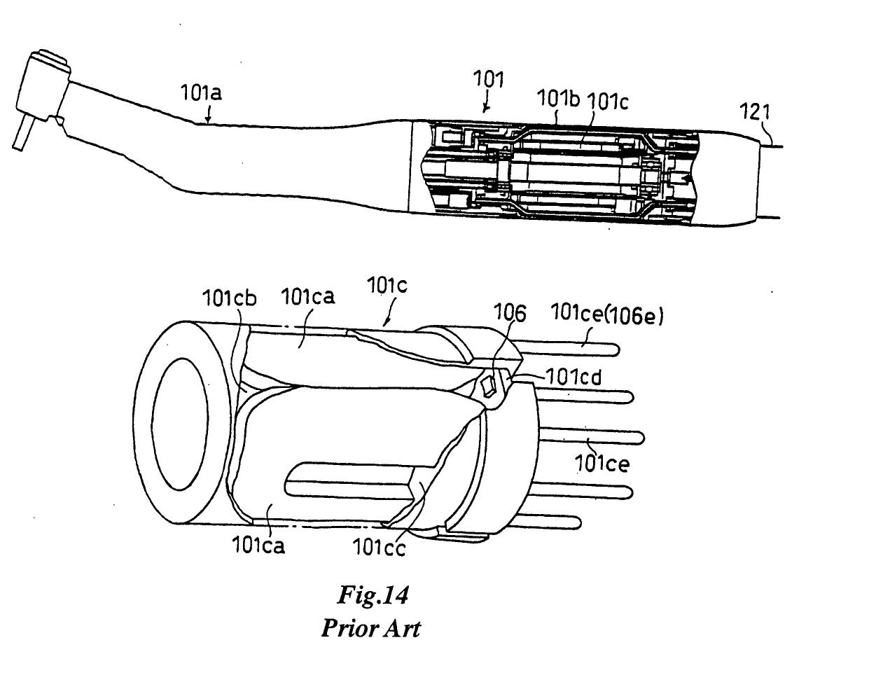

The instrument 1G which is a connection assembly with communication function has the same hardware construction as the instrument 101 shown in the prior art in FIG. 14.

Namely, a multi-junction connection part 21, a head 11a, a middle portion 11b, a stator 11c of a small motor, an inner cylinder 11cb, a coil 11ca, a board 11cd, resin 11cc, a connection terminal 11ce and a connection terminal 16e of the instrument 11 have the same construction as the multi-junction connection part 121, the head 101a, the middle portion 101b, the stator 101c of the small motor, the inner cylinder 101cb, the coil 101ca, the board 101cd, the resin 101 cc, the connection terminal 101ce and the connection terminal 106e of the prior instrument 101 in FIG. 14 respectively.

Only the communication means 6 is different from the identification signal output means 106 in the prior art and is constructed such that information on the connection assembly including functional information on the function to be realized by the instrument 1G in addition to the identification signal to identify the instrument 1G is communicated with the main body 10a upon being connected to the main body 10a as mentioned above.

Therefore, it isn't required for the main body 10a to store the functional information including a control program such as driving circuit, control circuit, and display mode and parameter required for the connected connection assembly so that the main body 10a can comply when the connection assembly is modified or changed or when a new connection assembly is connected.

When the resistive element as shown in FIG. 2, FIG. 3 and FIG. 4 is used as a communication means, it is preferable to use a corresponding table between the output of the resistive element and the functional information in order to obtain the functional information, not the identification information of the connection assembly, from the resistive element.

For example, if the resistive element is resistance and the functional information is the highest rotation number of the motor, the resistance value is set at 10 Ω, 20 Ω, 30 Ω . . . in order and the rotation speed is set to be 1000 rpm, 2000 rpm and 3000 rpm, . . . correspondingly. Further, back electromotive voltage constant value may be set at 1000 rpm/V, 2000 rpm/V, 300 rpm/V, . . . .

Accordingly, even if the set rotational number of the motor of the connected connection assembly is changed or revised at random, the setting rotational number isn't required to be memorized in the main body 10a, thereby obtaining a desirable setting rotation number which is the functional information of the connection assembly only by connecting the connection assembly.

On the other hand, the communication means which is the resistive element as shown in FIG. 2, FIG. 3 and FIG. 4 may be combined with the active communication means like FIG. 9.

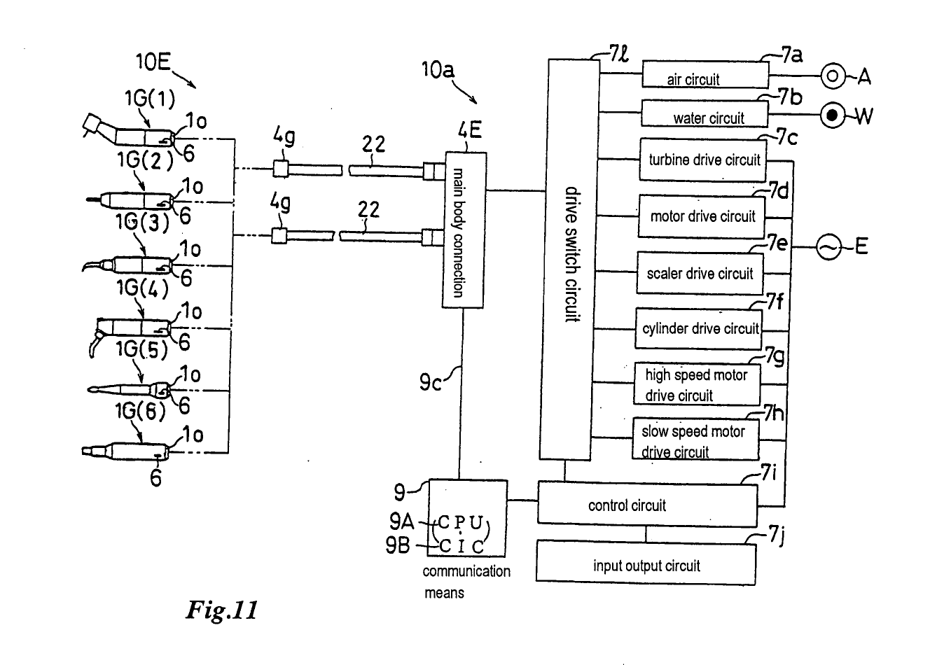

FIG. 11 shows a block diagram conceptionally showing one embodiment of the medical apparatus using the connection assembly as shown in FIG. 10.

In the medical apparatus 10E the instrument 1G (1-6) which is a connection assembly with communication function is detachably and exchangeably connected to a multi-junction connection part 4g provided at the tip end of plural flexible tubes 22 introduced from a connection part 4E of the main body 10a and works as a turbine handpiece 1G(1), a motor handpiece 1G(2), a scaler 1G(3), a three-way cylinder 1G(4), a high speed motor handpiece 1G(5) and a low speed motor handpiece 1G(6).

The apparatus body 10a includes an air circuit 7a for supplying air required for the instrument 1G upon receiving supply of compressed air from an air source A, a water circuit 7b for supplying water required for the instrument 1G upon receiving supply of water from a water source W, a turbine drive circuit 7c, a motor driving circuit 7d, a scaler driving circuit 7e, a cylinder driving circuit 7f, a high speed motor drive circuit 7d and a low speed motor circuit 7h for supplying driving power according to the kinds of the connected instruments 1G (1-6) upon receiving supply of outer power supply E, a control circuit for controlling the apparatus, an output means (see reference numeral 8a in FIG. 1) such as a liquid crystal display and a printer, and an input means (see reference numeral 8b in FIG. 1) such as a touch panel and a foot controller. And the apparatus body 10a is provided with an input/output circuit 7j for controlling them and an identification means 9.

A drive switch circuit 71 connects or disconnects the driving circuits 7c-7h provided for the instruments 1G (1-6) respectively and switches to connect the air circuit 7a and the water circuit 7b to the instruments 1G (1-6).

When any one of the instruments 1G (1-6) is connected to any one of the connection parts 4g, the apparatus body 10a reads the identification information and the functional information on the connected instrument 1G from the communication means 6 via the communication control means 9 to select the corresponding drive circuit. Simultaneously, corresponding to the connected instrument 1G based on the functional information, more detailed setting of the selected driving circuit, control of the control circuit 7i, a display mode control of the display means 8a of the input/output circuit 7j and the input mode of the input means 8b can be executed.

Further, if a control program is included in the functional information, the prior control program stored in the main body 10a may be revised using the included control program or the function to be achieved by the connected connection assembly may be realized by the updated control program by directly using the included program.

The communication means 6 executes communication output of ID code as identification information as explained referring to FIG. 5 so that the connected connection assembly is individually specified by means of the ID code in addition to the kinds of the connected connection assembly incorporating thereof. Namely, the communication means 6 can distinguish all the connection assembly respectively.

Therefore, it executes individual record management such as the frequency of usage, accumulated time of usage, and number of breakdown of each connection assembly and announce an alarm to the connection assembly which has been used for a predetermined time or controls disconnection of the drive control circuit 7k so as not to be used. Further, a connection assembly exclusive for each operator is prepared and when the other operator's connection assembly is connected by mistake, an alarm may be announced or the connection assembly becomes unavailable.

Following is an example of a connection assembly record management table for executing the above-mentioned individual record management used for the control circuit 7i of the medical apparatus 10.

<Connection Assembly Record Management Table>

| | | op- | | accumulated | |
|---|---|---|---|---|---|
| ID code | kind | erator | prior use | time | record |
| A0101001 | TH | AA | 000630/1500-1600 | 1500 H 03 M | K0 |
| A0101021 | TH | BB | 000703/1000-1100 | 1000 H 03 M | K1 |
| B0202001 | MH | AA | 000630/1600-1620 | 1600 H 20 M | K0 |
| C0303001 | SC | AA | 000620/1100-1110 | 1100 H 00 M | K1 |

In this table “TH” in “kind” is a turbine handpiece, “MH” is a motor handpiece, and “SC” is a scaler, in “prior use” the latest usage date is shown with year, month, and date in two digits, start time and end time shown with 24 hrs, in “accumulated time” the total used time of the connected assembly so far is shown with time (H) and minute (M), in record several prior records are shown, for example “K0” shows no breakdown and “K1” shows 1 breakdown.

When such a management table is prepared for the medical apparatus 10 and is revised each time the connection assembly is used, the individual record management of the connection assembly as mentioned above can be executed, thereby preventing an operator from using the other operator's connection assembly and exchanging the connection assembly which exceeds the predetermined time limit for usage. Therefore, minute management for usage of the connection assembly can be executed to contribute safer diagnosis and treatment.

The communication means actively outputs signals by itself so that it can send and receive signals wirelessly between the connection assembly and the main body, thereby reducing the number of the connection terminals of the connection part or constructing the connection assembly in a cordless manner as an independent type.

The communication means 6 of the connection assembly actively outputs identification signals so that the identification means 9 of the main body 10a is comprised of CPU (microcomputer element) 9A or CIC (communication integration element) 9B, thereby simplifying the construction of the apparatus. Further, if CPU (microcomputer element) has been already provided for the control circuit 7i of the main body 10a, the CPU of the control circuit 7i may be also used as the identification means 9 by using the program having the function of the identification means.

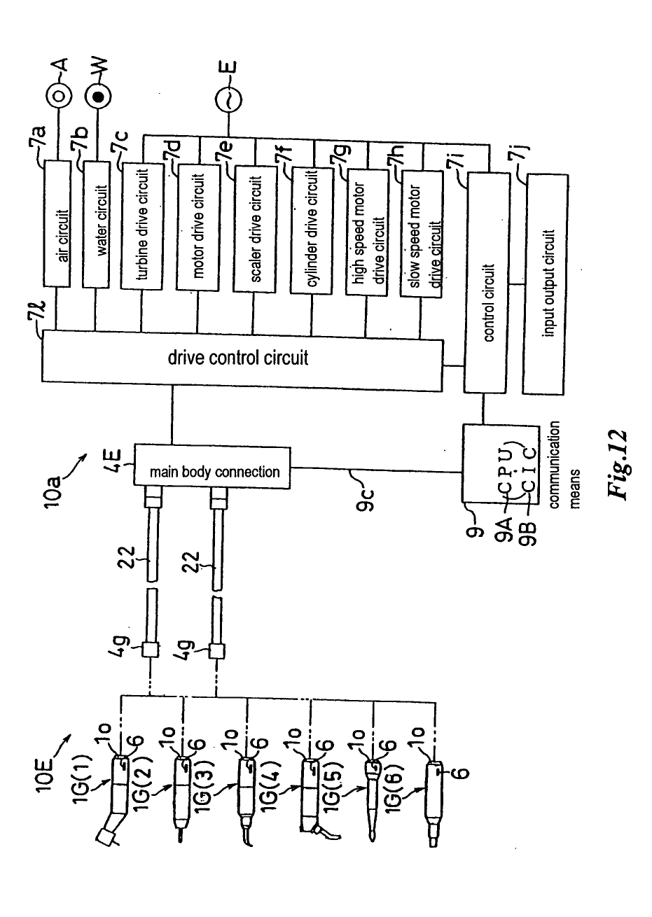

FIG. 12 shows a block diagram conceptionally showing other embodiment of the medical apparatus using the connection assembly with communication function according to the present invention.

The medical apparatus 10F is different from the apparatus 10E in FIG. 11 in that a connection assembly with communication function is an exclusive flexible tube 2 for connecting each instrument 1H (1-6) and an exclusive connection part 4h introduced from the connection part 4F of the main body 10a.

The exclusive flexible tube 2 incorporates a communication means 6 for communicating the information on each instrument 1H (1-6) to the main body 10a. The communication means 6 may be provided at the terminal end of the tube 2 as shown in the figure at the tip end or at the middle thereof.

The kinds of the instrument 1H (1-6) are the same as those of the medical apparatus 10E.

Also according to this construction, when any one of the instrument 1H (1-6) is connected to the exclusive connection part 4h via the exclusive tube 2, the identification information and functional information on the connected instrument 1H are read out of the communication means 6 by the communication control means 9 in the main body 10a, and corresponding drive circuit is selected. Simultaneously corresponding to the connected instrument 1H based on the functional information, more detail setting of the selected drive circuit, control of the control circuit, and control of display mode of a display means 8a of the input/output circuit 7j and input mode of an input means 8b can be executed.

Even if the communication means 6 doesn't have autoclave resistance in this case, only the instruments is subjected in autoclave by separating the instrument 1H(1-6) from the tube 2. Further, if the instrument 1H(1-6) isn't provided with communication function, communication function may be achieved at a low price by changing a cheaper tube 2 with the one having communication function.

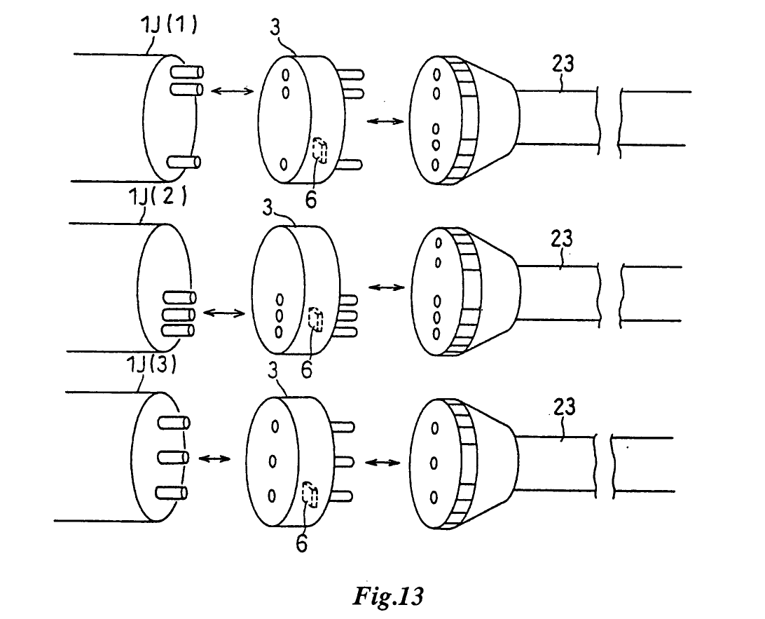

FIG. 13 shows a conceptoional diagram showing other embodiment of the connection assembly with communication function according to the present invention.

This embodiment is different from the embodiments which are explained hereinbefore in that the communication means 6 is incorporated in an adapter 3 interposed between an instrument 1J (1-3) and a flexible tube 23, not in the instrument 1J (1-3) or in the tube 23. Namely, the adapter 3 is constructed to be exclusive for each instrument 1J (1-3) so that the adapter 3 is combined with the instrument 1J (1-3) to realize communication function.

In such a construction, it is clear that the same effect as those in FIG. 11 and FIG. 12 can be achieved and the adapter 3 with communication function can solve the problem of autoclave of the instrument and can realize the instrument with communication function at low price like the tube 2 with communication function shown in FIG. 12.

The tube in FIG. 12 and the adapter in FIG. 13 can be constructed as a multi-junction connection and in such a case the communication means incorporated therein can realize communication more effectively.

1. A connection assembly detachably connected to a main body of a medical apparatus for use in diagnosis and treatment, wherein said connection assembly has a communication means for sending and receiving the information on said connection body to and from said medical apparatus.

2. The connection assembly as set forth in claim 1, wherein said communication means is comprised of a serial or parallel output type communication means.

3. The connection assembly as set forth in claim 1 or 2, wherein said communication means has a storage means for memorizing and storing the information on said connection assembly.

4. The connection assembly as set forth in claim 1 or 2, wherein a microcomputer element or a communication integration element is used as said communication means.

5. The connection assembly as set forth in claim 1, wherein said information is an identification information for identifying said connection assembly and/or a functional information on the function achieved by said connection assembly.

6. The connection assembly as set forth in claim 1 or 2, further comprising an identification signal output means with nonvolatile storage means, wherein any one of serial data, voltage level signals of which wave height value is varied at a predetermined repetition cycle, frequency identification signals of which frequency is varied is used as an identification signal from said identification signal output means, based on the data stored in said nonvolatile storage means.

7. The connection assembly as set forth in claim 1 or 2, further comprising a connection part for detachably connecting said instrument assembly to said main body, wherein said connection part constitutes a multi junction connection.

8. The connection assembly as set forth in claim 1, wherein said communication means is a passive element electrically to be connected to said main body of said medical apparatus.

9. The connection assembly as set forth in claim 1 or 2, wherein said connection assembly includes a charging battery.

10. A medical apparatus for use in diagnosis and treatment in which a connection assembly is detachably connected to a main body of the medical apparatus, wherein:
said connection assembly is comprised of a communication means for sending and receiving information on said connection assembly to and from said main body of said medical apparatus, and wherein function to be achieved by said connection assembly is realized cooperating with said connection assembly by the information obtained from said communication means upon connecting said connection assembly to said main body of said medical apparatus.

11. The medical apparatus for use in diagnosis and treatment as set forth in claim 10, wherein a part of said function is achieved by setting a driving circuit or a control circuit corresponding to the connected connection assembly.

12. The medical apparatus for use in diagnosis and treatment as set forth in claim 10 or 11, wherein a part of said function is achieved by setting display mode of display means and/or input mode of input means such as touch panel corresponding to the connected connection assembly.

13. The medical apparatus for use in diagnosis and treatment as set forth in claim 10 or 11, wherein the management of usage history and the distinction of using operator of the specified connection assembly can be executed, when said connection assembly is specified based on the information obtained form said connection assembly.

14. The medical apparatus for use in diagnosis and treatment as set forth in claim 10 or 11, wherein said main body of said medical apparatus has a microcomputer element or a communication integration element as a communication means for said connection assembly to be connected.

15. The medical apparatus for use in diagnosis and treatment as set forth in claim 10 or 11, wherein wiring to a connection part for detachably connecting said connection assembly in said main body constitutes a multi-branch structure.

16. The medical apparatus for use in diagnosis and treatment as set forth in claim 10, wherein said connection assembly is the connection assembly with communication function as set forth in claim 5.
